(19) United States
(12) Patent Application Publication
(10) Pub. No.: US 2024/0165193 A1
(43) Pub. Date: May 23, 2024

(54) Treatment of Ascites

(71) Applicant: BioVie Inc., Santa Monica, CA (US)

(72) Inventors: Paolo Angeli, Padova (IT); Penelope Markham, Clifton, VA (US); Jonathan Adams, Chicago, IL (US)

(21) Appl. No.: 18/420,332

(22) Filed: Jan. 23, 2024

Related U.S. Application Data
(63) Continuation of application No. 17/831,556, filed on Jun. 3, 2022.
Application No. 17/831,556 is a continuation of application No. 16/379,446, filed on Apr. 9, 2019, now Pat. No. 11,364,277.
Application No. 16/379,446 is a continuation of application No. 15/491,613, filed on Apr. 19, 2017, now abandoned.
Application No. 15/491,613 is a continuation of application No. 15/198,050, filed on Jun. 30, 2016, now Pat. No. 9,655,945.
(60) Provisional application No. 62/186,638, filed on Jun. 30, 2015.
(60) Provisional application No. 62/267,510, filed on Dec. 15, 2015.
(60) Provisional application No. 62/321,558, filed on Apr. 12, 2016.

(51) Int. Cl. A61K 38/095; A61P 1/00; A61P 1/16

(52) U.S. Cl. CPC A61K 38/095; A61P 1/00; A61P 1/16

(57) ABSTRACT

A method for treating ascites patients by administering the peptide drug terlipressin by continuous infusion. The patients include those whose ascites condition has not progressed to hepatorenal syndrome (HRS). Administration may be accomplished with a continuous infusion pump.

### RELATED APPLICATIONS

This application claims the benefit of U.S. provisional patent application 62/321,558, filed Apr. 12, 2016, U.S. provisional patent application 62/267,510, filed Dec. 15, 2015, and U.S. provisional patent application 62/186,638, filed Jun. 30, 2015, each of which is incorporated by reference herein their entirety

### FIELD

The disclosure is directed to a method for treating ascites patients by administering the peptide drug terlipressin.

### BACKGROUND

Ascites is a frequent and life-threatening complication of advanced liver cirrhosis with an expected 40% mortality rate within two years of diagnosis. To date the US FDA has not approved any therapies specifically to treat ascites, although a few drugs (e.g., diuretics) are being used off-label with limited and temporary efficacy. Studies have shown that intravenous (IV) injections of terlipressin every 4 to 6 hours in hospitalized patients with type 1 hepatorenal syndrome (HRS) can save their lives. HRS is the beginning of renal failure and frequently occurs in patients with ascites that has become refractory to treatment with diuretics. Additionally, investigational studies have shown that IV injections of terlipressin every 4 to 6 hours in combination with diuretics may resolve refractory ascites in hospitalized patients and decrease the need for large volume paracentesis (ascites fluid withdrawal by needle). However these intermittent high-dose IV injections (typically 1 or 2 mg in a single dose) carry a high risk of side-effects. More recent studies with hospitalized HRS patients indicate that a continuous infusion of terlipressin can achieve similar efficacy to intermittent injections with a much better safety profile. However to date there have been no published studies of using a continuous low-dose infusion terlipressin to manage ascites in non-hospitalized patients with cirrhosis.

Accordingly, the inventors have identified a need in the art for a method to treat ascites patients on an outpatient basis and potentially avoid or delay the need for hospitalization due to HRS or other life-threatening complications.

### SUMMARY

In one aspect, the disclosure is directed to a method for treating a patient diagnosed with ascites due to liver cirrhosis. The method including administering terlipressin or salt thereof as a continuous infusion. The condition of the patient may not have progressed to HRS.

In another aspect, the disclosure is directed to a method for reducing the volume of ascitic fluid during a paracentesis procedure in an ascites patient. The method includes administering terlipressin or salt thereof as a continuous infusion.

In yet another aspect, the disclosure is directed to a method for reducing the number of monthly paracentesis procedures in an ascites patient. The method includes administering terlipressin or salt thereof as a continuous infusion.

Still further, the disclosure is directed to a method for improving renal function in an ascites patient. The method includes administering terlipressin or salt thereof as a continuous infusion. In various aspects, the improvement in renal function includes one or more of the following: a reduction in serum creatinine concentration, an increase in plasma sodium concentration, an increase in urinary sodium excretion, and a decrease in urea concentration in serum.

The disclosure is also directed to a method for correcting hyponatremia in an ascites patient. The method includes administering to the patient terlipressin or salt thereof as a continuous infusion.

In a further aspect, the disclosure is directed to a method for improving the health status of the ascites patient with liver cirrhosis due to hepatitis C. The method includes method comprising administering a hepatitis C antiviral medication in combination with administering terlipressin or salt thereof as a continuous infusion.

In another aspect, the disclosure is directed to a method of improving the Model for End-Stage Liver Disease (MELD) score of an ascites patient. The method includes administering terlipressin or salt thereof with a continuous infusion.

In each of the aspects of the invention, the condition of the patient may not have progressed to HRS. Also, the terlipressin dose may range from about 1.0 mg to about 12.0 mg per day, and the terlipressin dose may be escalated over the course of the therapy. In addition, the terlipressin may be administered for a time period of about 1 day to about 12 months. Further, the continuous terlipressin may be administered with an ambulatory infusion pump.

### DESCRIPTION

Terlipressin is a synthetic vasopressin that is approved in many countries outside of the United States to treat the life-threatening complications of cirrhosis, including hepatorenal syndrome (HRS) and esophageal bleeding (EVB). Its use is limited to the hospital setting due to its short half-life (26 minutes) (Nilsson, et al., 1990), necessitating its administration as an intravenous bolus usually every 4 to 6 hours. Additionally, terlipressin can cause side effects in up to 40% of patients. Severe side effects—including myocardial infarction, arrhythmia and intestinal infarction—can require discontinuation of treatment in up to 10% of the patients (Angeli, 2011). Indeed, due to the rapid vasoconstrictor properties, IV bolus dosed terlipressin must be used with caution in patients with severe asthma, severe hypertension, advanced atherosclerosis, cardiac dysrhythmias, and coronary insufficiency.

In one aspect, the disclosure is directed to the administering terlipressin or a salt thereof for the treatment of patients suffering from ascites due to, for example, advanced liver cirrhosis. These patients are typically non-hospitalized (or ambulatory) and may include patients whose condition has not progressed to type 2 HRS (ambulatory HRS patients) or type 1 HRS (requiring hospitalization). Treatment includes a continuous infusion of terlipressin by means of a pump device, typically a portable ambulatory pump, for a period of several hours, lasting up to days, weeks, or months. The treatment is effective at reducing or resolving ascites disease on, for most patients, an outpatient basis.

Patients with cirrhosis exhibiting type 1 hepatorenal syndrome (HRS-1) have been safely treated with terlipressin administered continuously. Dosage ranged from 2.0-12.0 mg per 24 hours (Angeli, et al., 2009: 2-12 mg/24 h; Gerbes, 2009: starting dose 3 mg/day; Robertson, et al., 2014: 3 mg/day; Ding, 2013: 4 mg/day; Cavallin 2015: 3-12 mg/day). However, none of these studies have either evaluated or reported an effect of terlipressin infusion on ascites burden or the effect of continuous infusion terlipressin on patients whose condition have not progressed to HRS.

Ambulatory pumps are commonly used to infuse parenteral drugs directly into the bloodstream via catheters to increase efficacy and/or decrease toxicity. This has been found to be safer than some approved terlipressin drug therapy that require the administration of terlipressin to hospitalized hepatorenal syndrome (HRS) patients and esophageal bleed (EVB) patients using slow bolus IV injections. Accordingly, in one aspect of the disclosure, terlipressin is administered continuously by a pump at a dosage rate of about 0.5 mg to about 20 mg every 24 hours, more particularly for example, about 1 mg to about 12 mg every 24 hours, more particularly for example, about 5 to about 15 mg every 24 hours, or for instance, about 1, 2, 3, 4, 5, 6, 7, 8, 9, 10, 11, 12, 13, 14, 15, 16, 17, 18, 19, or 20 mg every 24 hours. Administration can continue for, typically, at least about one day and may continue for about 12 months or longer as necessary to bridge a patient until a transplant is available. For example, the administration can continue for about 1 day, 2 days, 3 days, 4 days, 5 days, 6 days, one week, two weeks, three weeks, one month, two months, three months, six months, 9 months or twelve months. In some instances, the dose of terlipressin escalates over the course of the therapy. For example, patients may begin therapy at 2 mg/day, and be increased to 3 mg/day or up to 12 mg/day over the course of treatment.

Accordingly, in various aspects, the disclosure is directed to a method for treating a patient diagnosed with ascites due to liver cirrhosis. The method can improve renal function in an ascites patient and reduce the volume of ascitic fluid during paracentesis procedure in the patient. Still further, the method can be used for reducing the risk of spontaneous bacterial peritonitis, improving the Model for End-Stage Liver Disease (MELD) score of an ascites patient and/or correcting hyponatremia in an ascites patient. In another aspect, the method disclosed herein can be used in combination with hepatitis C antiviral medications to improve the health status of the ascites patient with liver cirrhosis due to hepatitis C. In each case terlipressin or salt thereof is administered with a continuous infusion pump. In each of these aspects, the patient's ascites condition may not have progressed to hepatorenal syndrome.

In addition, the determination of the presence, progression, or improvement of disease can be determined by measuring one or more of the following: serum creatinine concentration, plasma sodium concentration, urinary sodium excretion, and urea concentration in serum. For example, an improvement in renal function that indicates an improvement in disease condition includes one or more of the following: a reduction in serum creatinine concentration, an increase in plasma sodium concentration, an increase in urinary sodium excretion, a decrease in urea concentration in serum of disease.

The use of ambulatory pump delivery of continuous infusion of terlipressin would avoid the need for patient hospitalization and make such therapy available to the vast majority of ascites patients who have not yet been hospitalized for severe complications that often follow advanced ascites, such as post-paracentesis circulatory dysfunction, HRS, EVB, hepatic encephalopathy, spontaneous bacterial peritonitis and other life-threatening conditions.

### EXAMPLES

The following are provided for exemplification purposes only and are not intended to limit the scope of the disclosure described in broad terms above.

### Example 1: Treatment of Ascites with Continuous Infusion Pump Terlipressin Therapy

15 subjects that are to be confirmed to have ascites, but not type 1 or type 2 HRS, due to liver cirrhosis will be administered continuous low dose (escalating from 2.0 to 3.0 mg per 24 hours) terlipressin via ambulatory infusion pump. These patients are expected to experience a decrease the severity of ascites and the accumulation of ascites fluid over the course of treatment ranging from 1 day to 28 days. This method is also expected to reduce the number of paracentesis procedures required to remove ascitic fluid over a 28-day period, compared to the 28-day period prior to treatment inception, and some patients should avoid paracentesis altogether. Additionally the average amount of fluid withdrawn after beginning continuous infusion pump terlipressin therapy should be significantly less than prior to the start of treatment. Furthermore the improvement in patient health status can be achieved safely with no serious side effects. Accordingly, continuous infusion pump (CIP) terlipressin represents a potentially life-saving solution for these seriously ill patients who are still ambulatory (have not yet been administered to the hospital for treatment) and have not developed type 1 or type 2 HRS.

### Example 2: Treatment of Ascites with Continuous Infusion Pump Terlipressin Therapy

Six HRS patients treated with continuous infusion terlipressin were evaluated for improvement in acsites. All six patients had diuretic intractable or refractory ascites (5 of 6 with hyponatremia). The patients were evaluated for the following parameters before, during and after treatment: number of paracentesis procedures per month, volume of ascites removed, weight, serum sodium, urinary sodium excretion, serum creatinine, serum urea, and whether diuretics were included in the treatment regimen. None of the six patients had a complete set of data for all parameters. The effect of continuous infusion terlipressin on each parameter is presented in Tables 1-7.

Reduction in frequency of paracentesis and fluid volume during therapy

The average number of monthly paracentesis procedures decreased from three prior to initiation of continuous infusion therapy to two during therapy, and the average monthly ascites fluid volume removed was reduced by 55%.

TABLE 1

| | Max. | | | | | Volume Fluid | | |
|---|---|---|---|---|---|---|---|---|
| Patient # | Dose | Duration | Paracenteses/Month | | | Removed/Month (L) | | |
| M/F | (mg/day) | (days) | Before | During | % Change | Before | During | % Change |
| 1 M | 12 | 63 | 1 | 0 | −100% | — | — | — |
| 2 F | 12 | 195 | 8 | 6 | −25% | 80 | 42 | −48% |
| 3 M | 3 | 10 | 4 | 2 | −50% | 40 | 14 | −65% |
| 4 M | 10 | 11 | 2 | 3 | 50% | 14 | 9 | −36% |
| 5 F | 3 | 22 | 3 | 2 | −33% | 21 | 6 | −71% |
| 6 F | 2 | 12 | 1 | 0 | −100% | 2 | 0 | −100% |

| | | | | | | |
|---|---|---|---|---|---|---|
| Average (excludes patient #1): | 3 | 2 | −32% | 31 | 14 | −55% |

“—” indicates missing data

### Reduction in Body Weight During Therapy

Average body weight per patient, a proxy for ascitic fluid accumulation in the abdominal cavity, decreased by 11% or 9 kg (˜19.8 lbs).

TABLE 2

| | Max. Terli. | | | | | |
|---|---|---|---|---|---|---|
| Patient # | Dose | Duration | Body Weight (kg) | | | |
| M/F | (mg/day) | (days) | Before | During | % Change | After |
| 1 M | 12 | 63 | 83 | 74 | −11% | 74 |
| 2 F | 12 | 195 | 64 | 71 | 11% | — |
| 3 M | 3 | 10 | 128 | 99 | −23% | 128 |
| 4 M | 10 | 11 | 60 | — | — | — |
| 5 F | 3 | 22 | 71 | 64 | −10% | 77 |
| 6 F | 2 | 12 | 64 | 55 | −14% | 68 |

| | | | | |
|---|---|---|---|---|
| Average (excludes Patient #4): | 82 | 73 | −11% | 87 |

“—” indicates missing data

### Requirement for Diuretics for Effect on Ascites

During treatment, improvement of ascites was seen without diuretics in four of six patients.

TABLE 3

| | Treatment | | | % Change | | |
|---|---|---|---|---|---|---|
| | Max. Terli. | | | Paracentesis | Volume | |
| Patient # | Dose | Diuretics | Diuretics | per | Fluid | Body |
| M/F | (mg/day) | Before | During | Month | Removed | Weight |
| 1 M | 12 | A | A | −100% | — | −11% |
| 2 F | 12 | A | None | −25% | −48% | 11% |
| 3 M | 3 | F + A | None | −50% | −65% | −23% |
| 4 M | 10 | F + A | None | 50% | −36% | — |
| 5 F | 3 | A | None | −33% | −71% | −10% |
| 6 F | 2 | F + A | F + A | −100% | −100% | −14% |

| | | | |
|---|---|---|---|
| Average: | −32% | −55% | −11% |

F = furosemide; A = anti-aldosteronic drug.
“—” indicates missing data.

### Increase in Urinary Sodium Excretion During Therapy

The observed improvement in ascites and renal function was further supported by a substantial increase in excretion of sodium into the urine. The average urinary sodium increased from 7 to 127 mEq/24 h in three of sis patients with data recorded before and after starting continuous infusion terlipressin therapy.

TABLE 4

| | Max. Terli. | | Urinary Na over 24 hours | | |
|---|---|---|---|---|---|
| Patient | Dose | Duration | (mEq/24 hr) | | |
| # M/F | (mg/day) | (days) | Before | During | % Change |
| 1 M | 12 | 63 | 5 | 46 | 820% |
| 2 F | 12 | 195 | — | 301 | — |
| 3 M | 3 | 10 | — | — | — |
| 4 M | 10 | 11 | 1 | 20 | 1900% |
| 5 F | 3 | 22 | — | 33/140 | — |
| 6 F | 2 | 12 | 16 | 315 | 1869% |

| | | | |
|---|---|---|---|
| Average (excludes patients #2, #3, #5): | 7 | 127 | 1632% |

“—” indicates missing data

### Improvement in Plasma Sodium

Treatment with continuous infusion terlipressin corrected severe hyponatremia in two patients: Plasma Na increased by 15% in patient #4 and by 19% in patient #6. Importantly, after the cessation of therapy, plasma sodium remained normal in patient #6 (data “after therapy” available for one of the two patients).

TABLE 5

| | | | Plasma | | | |
|---|---|---|---|---|---|---|
| | Max. Terli. | | Sodium | | | |
| Patient # | Dose | Duration | (mEq/L) | | | |
| M/F | (mg/day) | (days) | Before | During | % Change | After |
| 1 M | 12 | 63 | 140 | 137 | −2% | — |
| 2 F | 12 | 195 | 125 | 128 | 2% | — |
| 3 M | 3 | 10 | 133 | 136 | 2% | 140 |
| 4 M | 10 | 11 | 123 | 141 | 15% | — |
| 5 F | 3 | 22 | 131 | 128 | −2% | — |
| 6 F | 2 | 12 | 118 | 140 | 19% | 131 |

| | | | | |
|---|---|---|---|---|
| Average: | 128 | 135 | 5% | 136 |

“—” indicates missing data

### Reduction in Blood Urea During Treatment

The concentration of urea in patients' blood serum decreased in all patients by an overall average of 45%. This increase in urea clearance is indicative of improved renal function.

TABLE 6

| | Max. Terli. | | | | | |
|---|---|---|---|---|---|---|
| Patient # | Dose | Duration | Serum Urea (mmol/L) | | | |
| M/F | (mg/day) | (days) | Before | During | % Change | After |
| 1 M | 12 | 63 | 31.1 | 8.8 | −72% | — |
| 2 F | 12 | 195 | 36.6 | 23.2 | −37% | — |
| 3 M | 3 | 10 | 17.0 | 9.1 | −46% | 10.8 |
| 4 M | 10 | 11 | 51.8 | 37.3 | −28% | — |
| 5 F | 3 | 22 | 6.4 | 5.3 | −17% | 10.5 |
| 6 F | 2 | 12 | 20.4 | 6.6 | −68% | 10.0 |

| | | | | |
|---|---|---|---|---|
| Average: | 27.2 | 15.1 | −45% | 10.4 |

“—” indicates missing data

### Reduction in Serum Creatinine

Levels of the metabolic waste product serum creatinine are indicative of renal health. An average decrease of 47% was seen in serum creatinine levels for the treated group of patients. This was consistent with the decrease in serum urea and indicates improved renal function, contributing to a decrease in ascites severity.

TABLE 7

| | Max. Terli. | | | | | |
|---|---|---|---|---|---|---|
| Patient # | Dose | Duration | Serum Creatinine (mmol/L) | | | |
| M/F | (mg/day) | (days) | Before | During | % Change | After |
| 1 M | 12 | 63 | 248 | 189 | −24% | — |
| 2 F | 12 | 195 | 383 | 208 | −46% | — |
| 3 M | 3 | 10 | 233 | 116 | −50% | 122 |
| 4 M | 10 | 11 | 319 | 104 | −67% | — |
| 5 F | 3 | 22 | 68 | 55 | −19% | 55 |
| 6 F | 2 | 12 | 195 | 90 | −54% | 137 |

| | | | | |
|---|---|---|---|---|
| Average: | 241 | 127 | −47% | 105 |

“—” indicates missing data

All references cited in this disclosure are incorporated herein by reference.

Nilsson, G. et al., 1990. Nilsson G, Lindblom P, OhlPharmacokinetics of Terlipressin After Single i.v. Doses to Healthy Volunteers. Drugs Under Experimental and Clinical Research, Volume 16, pp. 307-314.

Angeli, P., 2011. Terlipressin for Hepatorenal Syndrome: Novel Strategies and Future Perspectives. Frontiers of Gastrointestinal Research, Volume 28, pp. 189-197.

Angeli, P. et al., 2009. Terlipressin Given as Continous Intravenous Infusion Versus Terlipressin Given as Intravenous Boluses in the Treatment of Type 1 Hepatorenal Syndrome (HRS) in Patients with Cirrhosis. Journal of Hepatology, 50(Supplement 1), p. S73.

Gerbes AL, Huber E, Gülberg V. 2009 Terlipressin for hepatorenal syndrome: continuous infusion as an alternative to i.v. bolus administration. 2009 Gastroenterology. 137(3):1179; author reply 1179-81

Ding, C. et al., 2013. Hemodynamic effects of continuous versus bolus infusion of terlipressin for portal hypertension: A randomized comparison. Journal of Gastroenterology and Hepatology, 28(7), pp. 1242-1246.

Robertson, M. et al., 2014. Continuous outpatient terlipressin infusion for hepatorenal syndrome as a bridge to successful liver transplantation. Hepatology Mar 2014. Hepatology, Volume March, pp. 1-2.

Cavallin M, et. al., 2015 Terlipressin Plus Albumin Versus Midodrine and Octreotide Plus Albumin in the Treatment of Hepatorenal Syndrome: A Randomized Trial. Hepatology, 2015 (in press)

Fimiani, B. et al., 2011. The Use of Terlipressin in Cirrhotic Patients with Refractory Ascites and Normal Renal Function: A Multicentric Study. European Journal of Internal Medicine, Volume 22, pp. 587-590.

Krag, A. et al., 2007. Telipressin Improves Renal Function in Patients with Cirrhosis and Ascites Without Hepatorenal Syndrome. Hepatology, 46(6), pp. 1863-1871.

Although various specific embodiments of the present disclosure have been described herein, it is to be understood that the disclosure is not limited to those precise embodiments and that various changes or modifications can be affected therein by one skilled in the art without departing from the scope and spirit of the disclosure.

1-20. (canceled)

21. A method for improving disease progression in a patient with ascites due to liver cirrhosis comprising:
determining the progression of disease in the patient by measuring at least one of of serum creatinine concentration and urinary sodium excretion; and
administering a continuous infusion of terlipressin or salt thereof to the patient;
wherein the improvement comprises reducing the risk of at least one of hepatorenal syndrome (HRS), esophageal bleeding (EVB), hepatic encephalopathy (HE), and spontaneous bacterial peritonitis (SBP) in the patient.

22. The method of claim 21, wherein the progression of disease in the patient is determined by measuring an elevated concentration of serum creatinine.

23. The method or claim 21, wherein the progression of disease in the patient is determined by measuring a decreased concentration of urinary sodium excretion.

24. The method of claim 21, wherein the progression of disease in the patient is determined by measuring an elevated concentration of serum creatinine and a decreased concentration of urinary sodium excretion.

25. The method of claim 21, wherein the administering is conducted at a continuous infusion dose of about 2.0 mg to about 12.0 mg per day.

26. The method of claim 21, wherein the administering is conducted at a continuous infusion dose of about 2.0 mg to about 12.0 mg per day for a duration of about one day to about twelve months.

27. The method of claim 21, wherein the continuous infusion is about 3 mg of terlipressin per day.

28. The method of claim 21, wherein the continuous infusion of terlipressin is administered with an ambulatory infusion pump.

29. The method of claim 21, wherein the administration is provided on an out-patient basis.

30. The method of claim 21, the improvement comprises reduced incidences of at least two of hepatorenal syndrome (HRS), esophageal bleeding (EVB), hepatic encephalopathy (HE), and spontaneous bacterial peritonitis (SBP) in the patient.

31. The method of claim 21, wherein the improvement further comprises a reduction in serum creatinine concentration during administration of the continuous infusion of terlipressin.

32. The method of claim 21, wherein the improvement further comprises a reduction in serum creatinine concentration after administration of the continuous infusion of terlipressin.

33. The method of claim 1, wherein the improvement further comprises an increase in urinary sodium excretion during administration of the continuous infusion of terlipressin.

34. The method of claim 21, wherein the improvement further comprises an increase in urinary sodium excretion after administration of the continuous infusion of terlipressin.

35. The method of claim 21, wherein the improvement further comprises reduction in the frequency of paracentesis procedures in a given time period after beginning administration of the continuous infusion of terlipressin.

36. The method of claim 21, wherein the improvement further comprises reduction in the volume of ascitic fluid that is removed per paracentesis procedure after beginning the administration of the continuous infusion of terlipressin.

37. The method of claim 21, wherein the improvement further comprises a reduction in patient weight during administration of the continuous infusion of terlipressin.

38. A method for delaying progression of advanced liver cirrhosis in a patient comprising:
determining a progression of liver cirrhosis in the patient by measuring at least one of a serum creatinine concentration and a urinary sodium excretion; and
administering a continuous infusion of terlipressin or salt thereof to the patient at risk for progression of advanced liver cirrhosis;
wherein the method reduces the risk of at least one of hepatorenal syndrome (HRS), esophageal bleeding (EVB), hepatic encephalopathy (HE), and spontaneous bacterial peritonitis (SBP) in the patient.

39. A method for avoiding hospitalization for esophageal bleeding (EVB), hepatic encephalopathy (HE), and spontaneous bacterial peritonitis (SBP) in patient with advanced ascites, the method comprising:
evaluating a patient for the progression of disease by measuring a patient's a serum creatinine concentration and a urinary sodium excretion;
administering a continuous infusion of terlipressin or salt thereof to patients with advanced acites.

40. A method for delaying progression of advanced liver cirrhosis in a patient comprising:
determining a progression of liver cirrhosis in the patient by measuring at least one of a serum creatinine concentration and a urinary sodium excretion; and
administering a continuous infusion of terlipressin or salt thereof to the patient at risk for progression to advanced liver cirrhosis;
wherein the method reduces the risk of at least one of esophageal bleeding (EVB), hepatic encephalopathy (HE), and spontaneous bacterial peritonitis (SBP) in the patient.
